(19) United States
(12) Patent Application Publication
(10) Pub. No.: US 2014/0366786 A1
(43) Pub. Date: Dec. 18, 2014

(54) DIFFERENTIAL MATURING REFUGE AND METHODS THEREOF

(71) Applicant: Monsanto Technology LLC, St. Louis, MO (US)

(72) Inventors: Matthew Wyatt Carroll, St. Louis, MO (US); Paula Ann Price, St. Louis, MO (US); Christopher L. Schuster, St. Louis, MO (US); William John Moar, St. Louis, MO (US); Graham Philip Head, St. Louis, MO (US); Scott Corey Johnson, Wildwood, MO (US); Chad Michael Peters, St. Louis, MO (US)

(21) Appl. No.: 14/304,715

(22) Filed: Jun. 13, 2014

Related U.S. Application Data
(60) Provisional application No. 61/835,470, filed on Jun. 14, 2013.

(51) Int. Cl. C12N 15/82; A01C 21/00

(52) U.S. Cl. CPC C12N 15/8286; A01C 21/00
USPC 111/200; 800/302; 47/58.1 FV

(57) ABSTRACT

The present invention provides a seed blend comprising pesticidal seed and refuge seed, wherein plants grown from said pesticidal seed type do not pollinate plants grown from said refuge seed type when the seed blend is planted. In some embodiments, the refuge crop reproductively matures at a different time relative to the time at which the pesticidal crop reproductively matures. Methods for deploying the seed blend and for reducing cross-pollination between plants grown from the pesticidal seed and refuge seed are also provided.

### REFERENCE TO RELATED APPLICATIONS

This application claims the benefit of U.S. provisional application No. 61/835,470 filed Jun. 14, 2013, which is herein incorporated by reference in its entirety.

### FIELD OF THE INVENTION

The invention relates to the control of pests that cause damage to crop plants through feeding. The invention also relates to the management of development of pest resistance to a pesticidal agent. In particular, the invention relates to seed blends and methods of deploying a refuge crop accompanying a transgenic-pesticidal crop.

### BACKGROUND OF THE INVENTION

Government regulatory agencies on a country-by-country basis require that transgenic-pesticidal crops (also referred to as “PIP” crops, for plant-incorporated protectants) be planted with a refuge crop. Refuge crops allow susceptible pest populations to survive, thereby (i) delaying the evolution of resistant pest progeny and (ii) improving durability of PIP crops.

In standard refuge practice, refuge plants can be cross-pollinated by pollen from PIP plants. As a result, developing progeny seed on a refuge plant can express the pesticidal transgene or PIP thereby affecting the survivorship of susceptible pests that feed on such seeds and decreasing the utility of the refuge crop.

### SUMMARY OF THE INVENTION

This invention relates to a new type of seed blend and methods for deploying a refuge crop. Relative to standard refuge practices, the present invention provides a refuge that results in increased survival of susceptible pests, thereby (i) delaying the evolution of resistant pest progeny and (ii) improving durability of PIP crops.

In one aspect, the invention provides a novel corn seed blend comprising (a) a first corn seed type comprising a first reproductive maturity rating and a transgene; and (b) a second corn seed type comprising a second reproductive maturity rating different from the first reproductive maturity rating that lacks said transgene, wherein the second reproductive maturity rating differs from the first sexual maturity rating by at least 3 days. In one embodiment, plants grown from the second corn seed type are substantially unpollinated by plants grown from the first corn seed type and in another embodiment, plants grown from the first corn seed type are substantially unpollinated by plants grown from the second corn seed type. In yet another embodiment, a plant grown from the second seed type sexually matures at a time different from a plant grown from the first seed type due to a difference in time to silk or time to mid-silk.

In a further embodiment, the second corn seed type may comprise another transgene, such as a transgene encoding a toxin effective against a pest different than the pest for which the transgene product of the first corn seed type is effective against, or a transgene encoding an herbicide. In certain embodiments of the invention, the transgene expresses a pesticide (such as a fungicide, insecticide, or PIP, such as Bt or RNA).

In one embodiment, a corn ear produced by plants grown from the second corn seed type exhibit a reduced number of seed comprising the transgene when compared to a control corn seed blend comprising seed types rated to reproductively mature contemporaneously.

In other embodiments, the ratio of the first corn seed type to the second corn seed type is from about 70:30 to about 99:1, from about 80:20 to about 97:3, or from about 90:10 to about 95:5. For instance, the ratio may be about 70:30, 71:29, 72:28, 73:27, 74:26, 75:25, 76:24, 77:23, 78:22, 79:21, 80:20, 81:19, 82:18, 83:17, 84:16, 85:15, 86:14, 87:13, 88:12, 89:11, 90:10, 91:9, 92:8, 93:7, 94:6, 95:5, 96:4, 97:3, 98:2, or 99:1.

In a further embodiment, the relative maturity rating for plants grown from seed of the second corn seed type may be at least 3, 4, 5, 6, 7, 8, 9, 10, or 11 days earlier or later than the relative maturity rating for plants grown from seed of the first corn seed type. In still further embodiments, the first corn seed type comprises an event selected from the group consisting of DP-186165-2, DP-186169-6, DP-187156-3, DP 4114, MIR162, MIR 604, Bt 176, TC1507, DAS-06275-8, DAS-59122-7, Bt11, 5307, MON810, MON89034, MON88017, ZM_S295399, MON87411, MON853, and MON863. In other embodiments, the first corn seed type comprises a transgene encoding for a pesticidal product selected from the group consisting of VIP3A, VIP3Aa, Cry1A.105, Cry2Ab, Cry1F, Cry1A, Cry1Ab, Cry1Ac, Cry34/35, Cry34/35Ab1, Cry34Ab1, Cry35Ab1, Cry3A, mCry3A, eCry3.1Ab, Cry3Bb, Dv49 dsRNA, and Dv_Snf7o.

In another aspect, the invention provides a field of plants grown from the corn seed blend of the present invention.

In still another aspect the invention provides a method of reducing the incidence of pollination between plants grown from different seed types in a corn seed blend, the method comprising: a) providing a corn seed blend comprising (i) a first corn seed type comprising a first reproductive maturity rating and a transgene and (ii) a second corn seed type comprising a second reproductive maturity rating different from the first reproductive maturity rating that lacks said transgene; and (b) planting the corn seed blend, wherein the second reproductive maturity rating differs from the first sexual maturity rating by at least 3 days.

In one embodiment, plants grown from said second corn seed type are substantially unpollinated by plants grown from said first corn seed type and in another embodiment, plants grown from said first corn seed type are substantially unpollinated by plants grown from said second corn seed type.

In another embodiment, a corn ear produced by plants grown from said second corn seed type exhibit a reduced number of seed comprising said transgene when compared to a control corn seed blend comprising seed types rated to reproductively mature contemporaneously. In yet another embodiment, the ratio of seed of said first corn seed type to seed of said second corn seed type in said seed blend is selected from the group consisting of from about 70:30 to about 99:1, from about 80:20 to about 97:3, and from about 90:10 to about 95:5. In still another embodiment, the pollen shed period of plants grown from seed of said first seed type does not substantially overlap with the mid-silk timing of plants grown from seed of said second seed type.

In further embodiments, the first corn seed type comprises an event selected from the group consisting of DP-186165-2, DP-186169-6, DP-187156-3, DP 4114, MIR162, MIR 604, Bt 176, TC1507, DAS-06275-8, DAS-59122-7, Bt11, 5307, MON810, MON89034, MON88017, ZM_S295399, MON87411, MON853, and MON863. In other embodiments, the first corn seed type comprises a transgene encoding for a pesticidal product selected from the group consisting of VIP3A, VIP3Aa, Cry1A.105, Cry2Ab, Cry1F, Cry1A, Cry1Ab, Cry1Ac, Cry34/35, Cry34/35Ab1, Cry34Ab1, Cry35Ab1, Cry3A, mCry3A, eCry3.1Ab, Cry3Bb, Dv49 dsRNA, and Dv_Snf7o. In an additional embodiment, the pesticidal agent is active against Helicoverpa zea, and the reduction occurs at the tip portion of the corn ears.

In another aspect, the invention provides a method of reducing the incidence of pollination between plants grown from different seed types in a corn seed blend comprising applying a treatment to plants grown from said corn seed blend, wherein (a) plants grown from said first corn seed type comprise a gene or gene product that interacts with said treatment and plants grown from said second corn seed type lack said gene or gene product, and wherein said interaction delays or accelerates time to reproductive maturity of plants; or (b) plants grown from said second corn seed type comprise a gene or gene product that interacts with said treatment and plants grown from said first corn seed type lack said gene or gene product, and wherein said interaction delays or accelerates time to reproductive maturity of plants.

In a still further aspect, the invention provides a method for ensuring compliance with government regulations for planting a refuge crop with or alongside of a transgenic crop, said method comprising: (a) providing a corn seed blend comprising (i) a first corn seed type comprising a first reproductive maturity rating and a transgene and (ii) a second corn seed type comprising a second reproductive maturity rating different from the first reproductive maturity rating that lacks said transgene; and (b) planting the corn seed blend, wherein the second reproductive maturity rating differs from the first sexual maturity rating by at least 3 days.

### DESCRIPTION OF THE DRAWINGS

FIG. 1A-FIG. 1C illustrate field configurations representing refuge-integrated (1A), no refuge (1B), and block refuge (1C) configurations. Plant-incorporated-protectant (PIP) planting locations are represented by “1st”, and refuge planting locations are represented by “2nd”.

### DETAILED DESCRIPTION

The present invention provides compositions and methods for reducing the risk of insect resistance development in cultivated insecticidal crops. In particular, the invention relates to improved insect resistance management practices, such as novel seed blends, and improved methods for deploying a crop refuge. Such improvements may be provided, for instance, by reducing cross-pollination of refuge crop plants by plant-incorporated-protectant (PIP) crop plants in a refuge-integrated field.

Insect Resistance Management (IRM) provides for reducing the likelihood of development of resistance to one or more recombinant traits, such as a PIP, that is present within a recombinant plant. One approach for achieving resistance management is through a seed mix or seed blend refuge composition, e.g., refuge-in-a-bag (RIB). However, cross-pollination of a refuge crop plant by transgene-containing pollen from a PIP crop plant can result in production of progeny seed on the refuge crop plants expressing the pesticidal transgene. Such PIP progeny seed produced on refuge plants may impact the survivorship of susceptible pests that feed upon the seed, thus diminishing the efficacy of the delay in evolution of pesticide resistant pests.

Reduction of the cross-pollination of refuge crop plants by PIP crop plants may therefore increase the production of susceptible insects in the refuge plant population of a field. In one embodiment, the present invention therefore provides a method of reducing cross-pollination of a refuge crop plant by PIP crop plant. Such reduction in cross-pollination may, for instance, be a reduction in cross-pollination of an individual refuge plant by an individual PIP plant, when both plants are grown from a seed blend. Such reduction in pollination may also be a reduction in cross-pollination of a population of refuge plants by a population of PIP plants.

In one embodiment, the invention provides a method for deploying refuge crops comprising planting in a field a seed blend comprising seed of a PIP crop population and seed of a refuge crop population, where the PIP crop population and refuge crop population reach reproductive maturity at different times in the field. For instance, plants of the PIP crop population may reproductively mature relatively earlier, relatively later, or relatively earlier and later, than plants of the refuge crop population. Such a seed blend can be described to have “differential sexual maturity ratings” between the two seed types. Plants in the PIP crop population and refuge population reaching reproductive maturity at different times may, in one embodiment, physiologically mature at the same time or may physiologically mature at a relatively different time, such as relatively earlier, relatively later, or relatively earlier and later.

Relative maturing ratings, e.g., time to reproductive maturity or physiological maturity of a plant species, can be predicted to varying accuracy depending on plant species and knowledge of parental maturing rates. Maturing ratings relative to female and male parts of plant species can also be predicted with precision, especially with highly cultivated crop species such as field corn.

In certain embodiments, the PIP seed and the refuge seed are provided as a seed mixture or seed blend. The invention therefore provides a novel seed mixture, a novel seed blend, or a novel improved refuge-in-a bag (iRIB). Seeds in such a mixture may be planted to produce a refuge-integrated field. The invention therefore further provides a refuge-integrated field resulting from the deployment seed mixtures or seed blends according to the invention.

A refuge-integrated field planted in accordance with the present invention may result in a population of refuge crop plants that is reproductively asynchronous to the population of PIP crop plants deployed therewith. Developing progeny seed produced on the refuge crop may therefore be substantially pollinated (self- and/or cross-pollinated) by the refuge crop and substantially unpollinated by the PIP crop. Therefore, compared to a refuge-integrated field deployed from a standard refuge seed blend where refuge crops and PIP crops sexually mature contemporaneously, the refuge-integrated field in accordance with the present invention may exhibit a reduction in cross-pollination of refuge plants by PIP plants, thereby increasing the surviving population of susceptible insects feeding on the progeny seed of refuge plants.

In another embodiment, the reduction in cross-pollination provided by the present invention can result in a spatially relevant reduction of transgene transfer in a kernel assemblage, such as an ear of corn. For example, the differential maturity ratings of seeds in the blend may provide that, if transgene transfer due to cross-pollination occurs at all, it is limited to a particular spatial orientation on the resulting kernel assemblage, such as a location on the assemblage away from insect larvae feeding sites. For instance, in corn fields Helicoverpa zea (corn earworm, CEW) typically start feeding at the tip or stalk distal portion of a corn ear. Therefore, a reduction of cross-pollination may result in corn ears on refuge plants that do not comprise corn kernels containing the transgene on the distal portion, or tip of the ear, thus resulting in increased survival of susceptible CEW. Similarly, a reduction of cross-pollination resulting in corn ears on refuge plants with corn kernels not containing the transgene on bottom the portion of the ear, or the portion proximal to the stalk, can result in susceptible corn borer (Ostrinia nubilalis) or fall armyworm (Spodoptera frugiperda) survival.

Novel seed blends in accordance with the present invention comprise at least a first PIP seed, which contains at least a first transgene, and at least one type of refuge plant seed. The refuge plant seed can be uniform in nature, in that it is composed of a single type of seed from a single variety of plant, or can be non-uniform in nature and consist of two or more varieties of plant. In one embodiment, the refuge seed is similar in variety (or agronomic characteristics) to the first transgenic crop seed. In another embodiment, the refuge seed is of a variety that is rated to reach reproductive maturity at a different respective time in the field than that of the transgenic crop seed.

The refuge seed can be non-transgenic or can be transgenic. A refuge seed that is a transgenic seed can contain any transgene so long as it is not the insecticidal transgene that is present in the first transgenic crop seed. In certain embodiments, the transgene product in a transgenic refuge seed has insecticidal activity against a different pest or against the same pest, but by a different mode of action, than the transgene product in the PIP crop seed. In other embodiments, the transgene in a transgenic refuge seed is an insecticidal gene, a herbicide tolerance gene, a fungicide tolerance gene, a fragment of an insect gene, or the like or a combination thereof. Refuge seeds may be grown into plants that act as a refuge for pests that either feed directly on a particular crop species, or other pests, the presence of which within the local proximity of a particular crop species, results in the damage, decrease in viability, infertility, or decrease in yield of a crop produced from such crop species.

In various embodiments, the contribution of refuge crop seed to the seed mixture or seed blend can be measured by percentage weight or count of refuge seed to total weight or count of the seed mixture. Seed mixtures in accordance with the present invention for deployment in a field may comprise from about 1% to about 50% refuge crop seed, from about 1% to about 10% refuge crop seed, or from about 5% to about 10% refuge crop seed. Refuge seed contribution can therefore be about 50%, 45%, 40%, 35%, 31%, 30%, 29%, 28%, 27%, 26%, 25%, 24%, 23%, 22%, 21%, 20%, 19%, 18%, 17%, 16%, 15%, 14.5%, 14%, 13%, 12%, 11%, 10%, 9%, 8%, 7%, 6%, 5%, 4%, 3%, 2%, or 1% total weight or count of the seed mixture. That is, the PIP crop seed might comprise about 50%, 55%, 60%, 65%, 70%, 75%, 80%, 85%, 85.5%, 90%, 95% or up to about 99% of the seed mixture. The respective PIP:refuge ratio (ratio of transgenic plant incorporated protectant seed to refuge seed) in seed mixtures of the present invention may be about 50:50 to about 99:1, about 70:30 to about 99:1, about 80:20 to about 97:3, or about 90:10 to about 95:5, and any integer or fraction ratio in between, for example, about 50:50, 55:45, 60:40, 65:34, 70:30, 75:25, 80:20, 85:15, 85.5:14.5, 90:10, 95:5, 97:3, or 99:1.

In one embodiment, the plant species of the PIP crop and refuge crop may be of the seed-bearing type. In other embodiments these plants may be capable of harboring a PIP. For instance, the plants may be selected from the group consisting of maize, corn, field corn, sweet corn, soybean, cotton, canola, wheat, rice, alfalfa, tobacco, sunflower, coffee species, tea species, grapes, plum, papaya, squash, flax, or tree species (poplar, aspen, sweetgum, eucalyptus, or spruce). In yet another embodiment, the plants may be capable of being subject to infestation and damage by a pest against which the PIP is pesticidal. In certain embodiments, plants of the present invention are protected against pests that contain in their diet parts of such plants, such as the developing embryos of the plant, e.g., maize ears, corn kernels, soybeans, or cotton seed.

Substantially unpollinated, as used herein can refer to a seed assemblage produced by a refuge crop that exhibits 50%, 49%, 48%, 47%, 46%, 45%, 44%, 43%, 42%, 41%, 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, 1%, or 0% of its kernels or seed having the transgene responsible for the PIP activity.

A measurable reduction in cross-pollination between PIP crop and refuge crop plants grown from a seed blend of the present invention, as compared to that of a seed blend in which the PIP crop and refuge crop plants reproductively mature contemporaneously will further delay insect resistance development. The reduction in number of PIP kernels in the kernel assemblage of refuge plants of the present invention as compared to those on refuge plants grown from a standard seed blend comprising PIP crop and refuge crop seed that reproductively mature contemporaneously can be described in terms of -fold reduction. This reduction can be, for example, a 1.05-fold, or 1.10-fold, or 1.18-fold, or 1.25-fold, or 1.30-fold, or 1.40-fold, or 1.50-fold, or 1.70-fold, or 1.80-fold, or 2.0-fold, or any fold greater than one (1).

For example, plants grown from a standard seed blend comprising PIP crop and refuge crop seed that are rated to reproductively mature contemporaneously can exhibit an average 75% PIP kernels on a refuge kernel assemblage, and plants grown a seed blend in accordance with the present invention can exhibit an average 50% PIP kernels on a refuge kernel assemblage; which is equivalent to a 1.5-fold reduction in pollination of refuge plants by PIP plants.

In another example, a refuge kernel assemblage produced from a standard seed blend comprising PIP crop and refuge crop seed that are rated to reproductively mature contemporaneously can exhibit 25 PIP kernels on the refuge kernel assemblage, and a refuge kernel assemblage produced from a seed blend in accordance with the present invention can exhibit 5 PIP kernels on the refuge kernel assemblage; which is equivalent to a 5-fold reduction in pollination of refuge plants by PIP plants.

In yet another example, a population of refuge kernel assemblages produced from a standard seed blend comprising PIP crop and refuge crop seed that are rated to reproductively mature contemporaneously can exhibit pest survival on 10% of the population of refuge kernel assemblages, and a population of refuge kernel assemblages produced from a seed blend in accordance with the present invention can exhibit pest survival on 50% of the population of refuge kernel assemblages; which is equivalent to a 5-fold increase in pest survival.

In another aspect of the invention, the relative difference in reproductive maturity between the PIP crop seed and the refuge crop seed may be a result of treating either the PIP crop seed or the refuge crop seed with a formulation to delay or accelerate plant growth rate to reproductive maturity. In one embodiment, treatment with the formulation may occur prior to planting, such as a seed treatment, or may occur after planting the seed, such as a soil drench or foliar spray. In certain embodiments, such treatment formulations that delay developmental timing may include plant hormone growth regulators, e.g., abscisic acid (ABA) and BIONIK (a formulation of 25% s-abscisic acid (s-ABA)).

In another embodiment, the PIP crop of the present invention may harbor a gene or gene product that interacts with the treatment resulting in a delay or acceleration in the time to reproductive maturity, while the refuge crop lacks the gene or gene product. Alternatively, the refuge crop may comprise the gene or gene product, while the PIP crop lacks the gene or gene product. In various embodiments, the gene or gene product may be a transgene encoding an herbicide tolerant protein product that protects the crop from reproductive delay due to herbicide treatment. Alternatively, the gene or gene product may be an endogenous plant reproductive pathway gene that is sensitive to RNA treatment, e.g., down-regulating the maize flowering time gene “delayed flowering1” (dlf1 is required for timely promotion of the floral transition) by RNA treatment.

In a further embodiment, the seed mixture includes PIP crop seed having a uniform relative reproductive maturity rating, and where the difference between the relative reproductive maturity rating of the refuge crop seed and the PIP crop seed is at least one day. For instance, the difference between the relative maturity of the refuge crop seed and the PIP crop seed may be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or 31 day or greater. In certain embodiments, the pollen shed period of plants grown from the PIP crop seed does not overlap or substantially overlap with the silking period or the mid-silk timing of plants grown from the refuge crop seed. An effective difference between the relative reproductive maturity of the refuge crop seed and of the PIP crop seed can depend on the insect feeding behavior, PIP efficacy, or PIP pollination pattern.

In certain embodiments, the PIP crop seed comprises a transgene expressing a pesticidal product selected from the group consisting of VIP3A, VIP3Aa, Cry1A.105, Cry2Ab, Cry1F, Cry1A, Cry1Ab, Cry1Ac, Cry34/35, Cry34/35Ab1, Cry34Ab1, Cry35Ab1, Cry3A, mCry3A, eCry3.1Ab, Cry3Bb, Dv49 dsRNA, and Dv_Snf7o. In still further embodiments, the PIP crop seed is a maize seed and harbors a transgenic event selected from the group consisting of DP-186165-2, DP-186169-6, DP-187156-3, DP 4114, MIR162, MIR 604, Bt 176, TC1507, DAS-06275-8, DAS-59122-7, BO 1, 5307, MON810, MON89034, MON88017, ZM_S295399, MON87411, MON853, and MON863.

In other embodiments, the PIP crop seed is a Glycine max seed and harbors a transgenic event selected from the group consisting of MON87701, MON89788, DP-063923-7, DP-068862-5, and DP-082117-3.

### DEFINITIONS

Unless defined otherwise, all technical and scientific terms used herein have the same meaning as commonly understood by one of ordinary skill in the art to which this invention belongs. The inventors do not intend to be limited to an example or to a variation of practice.

The term “field” refers to a cultivated expanse of land that a farmer uses to grow a crop species. A field ranges in size depending on crop species and purpose. In one embodiment, a maize field can include rows. Row spacing in a maize field can vary, e.g., from 15 inches to 22 inches to about 30 inches. Maize field rows can be planted at various rates, e.g., from about 12,000, 18,000, 24,000, 30,000, 34,000, 36,000, to 50,000 seed per acre; and can be planted at various lengths.

As used herein, the term “PIP” stands for “plant-incorporated protectant”, and can mean “toxin”, “pesticide”, “Bt” where the plant protectant is a protein from Bacillus thuringiensis, or “RNA” where the plant protectant is an RNA molecule. PIPs can be expressed from a transgenic event that comprises a transgene encoding the PIP.

A “refuge-integrated” field as used herein refers to a field containing a population of PIP plants and a population of refuge plants. PIP and refuge populations can be planted from a seed blend, such as a “refuge-in-a-bag” (RIB) or “improved RIB” (iRIB) seed blend. Such a field may include a first population PIP crop that harbors a PIP with pesticidal activity against a pest or a number of pests of the crop species, and a second population refuge crop that is not pesticidal against the pest or the number of pests of the crop species or is insecticidal against the same pest or number of pests, but through a different mode of action. As used herein, the first population PIP crop is grown from a “first seed type”, “toxin seed”, “pesticidal seed” or “PIP seed”, and the second population refuge crop is grown from a “second seed type” or “refuge seed”.

As used herein, the term “refuge” includes an isolated plant or plant population; including the plant parts, such as the seed, developing seed, or seed assemblage of the isolated refuge plant or refuge plant population. The refuge crop (plant population, isolated plant, or plant parts) functions as a dietary refuge or refugia for pest(s) under PIP control by nearby plants or plant parts comprising the PIP.

As used herein, the term “relative maturity rating” (RM) denotes the expected number of days after planting for a crop species to reach physiological maturity. For example, in maize crop species, crop varieties that share the same RM are rated to reach kernel black layer formation at about the same time and thus will typically be ready for ear harvest at the same time.

As used herein, the terms “reproductive maturity” and “sexual maturity” are used interchangeably. The terms “reproductive maturity rating” (RpM) and “sexual maturity rating” refer to the expected number of days after planting for a crop species to produce male or female reproductive structures, such as tassel and stigma, thereby reaching reproductive or sexual maturity. In hybrid corn seed production, RpM is predicted based on the observed parental developmental timing of reproductive tissue. RpM can be predicted precisely, for example, to within a 3 day range for a crop population of commercial hybrid seed. Plants grown from hybrid seed rated to reproductively mature at the same time can be expected to reproductively mature contemporaneously in the field. As used herein, when referring to a crop population of more than one plant grown in a field, the term “contemporaneous” means “substantially at the same time.”

In various embodiments, hybrid varieties are also contemplated that reach physiological maturity (RM) contemporaneously, but differ in timing with regard to reproductive (sexual) maturity (RpM). For example, maize varieties that exhibit physiological relative maturity ratings of 114 to 118 days, may exhibit timing to reproductive (sexual) maturity differing by up to six days.

Other methods to rate developmental time periods of hybrid maize varieties are based on thermal time, e.g., growing degree days (GDDs), growing degree units (GDUs), and heat units (HUs). For example, the accumulated GDUs between planting and silking, or between planting and pollen shed, are precisely estimated for hybrid maize varieties. Additionally, the silking and pollen shed periods of hybrid maize varieties are typically further resolved by percentage points from start of the period (0%) to end of the period (100%), with mid-phase of silking (S50; 50% of a population of maize plants predicted to have started silking) and mid-phase of pollen shed (P50; 50% of a population of maize plants predicted to have started shedding pollen) typically rated. In certain embodiments, the silking and pollen shed stage may be from about 1% to 100%, including about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, and 99%. In another embodiment, the tassel development stage for predicting RpM may be between T0 and T7, such as T0, T1, T2, T3, T4, T5, T6, and T7. Variations of rating systems are contemplated in the present invention.

As used herein, this difference in timing(s) between maturities of the two seed types can also be termed “differential” or “differential RM” or “differential maturing refuge”.

As used herein, the term “harboring” means “comprising”, “having”, or “transgenic for”, that is, a plant or plant cell harboring a transgene, Bt, or gene, refers to a plant or plant cell that comprises, or is transgenic for, the transgene, Bt, or gene.

As used herein, plants from the “same species” refers to seed-bearing plants which are reproductively compatible, that is, permitting breeding between plants of the species, including wild species.

As used herein, the tip of a corn ear can be identified when the ear is still attached to the stalk as the stalk distal portion of the corn ear. When unattached, the tip of a corn ear can be identified as the butt distal portion of the corn ear. In certain embodiments, the tip of the corn ear can comprise the stalk distal ½, ⅓, ⅕, 1/10, 1/20 of the ear.

The term “deploying” in the context of a field, may optionally include, e.g., preparing soil, treating seed, imaging seed, mixing seed, blending seed, planting seed, growing plants from the seed, applying water, fertilizing, applying plant protectants such as pesticides and fungicides, applying herbicides, applying desiccants or defoliants, and drying down for harvest of the crop. As used herein, “planting” seed includes “growing” plants from the seed. Unless otherwise noted, the rationales to performing such steps and the order in which they are performed would be known by one of ordinary skill in the art, e.g., a farmer.

### EXAMPLES

Those of skill in the art will appreciate the many advantages of the methods and compositions provided by the present invention. The following examples are included to demonstrate the preferred embodiments of the invention. It should be appreciated by those of skill in the art that the techniques disclosed in the examples that follow represent techniques discovered by the inventors to function well in the practice of the invention, and thus can be considered to constitute preferred modes for its practice. However, those of skill in the art should, in light of the present disclosure, appreciate that many changes can be made in the specific embodiments that are disclosed and still obtain a like or similar result without departing from the spirit and scope of the invention. All references cited herein are incorporated herein by reference to the extent that they supplement, explain, provide a background for, or teach methodology, techniques, or compositions employed herein.

### Example 1

This example illustrates a method to assess the refuge potential of a refuge plant population accompanying a PIP plant population in a field, where the PIP targets a pest that feeds on the developing progeny seeds in the field.

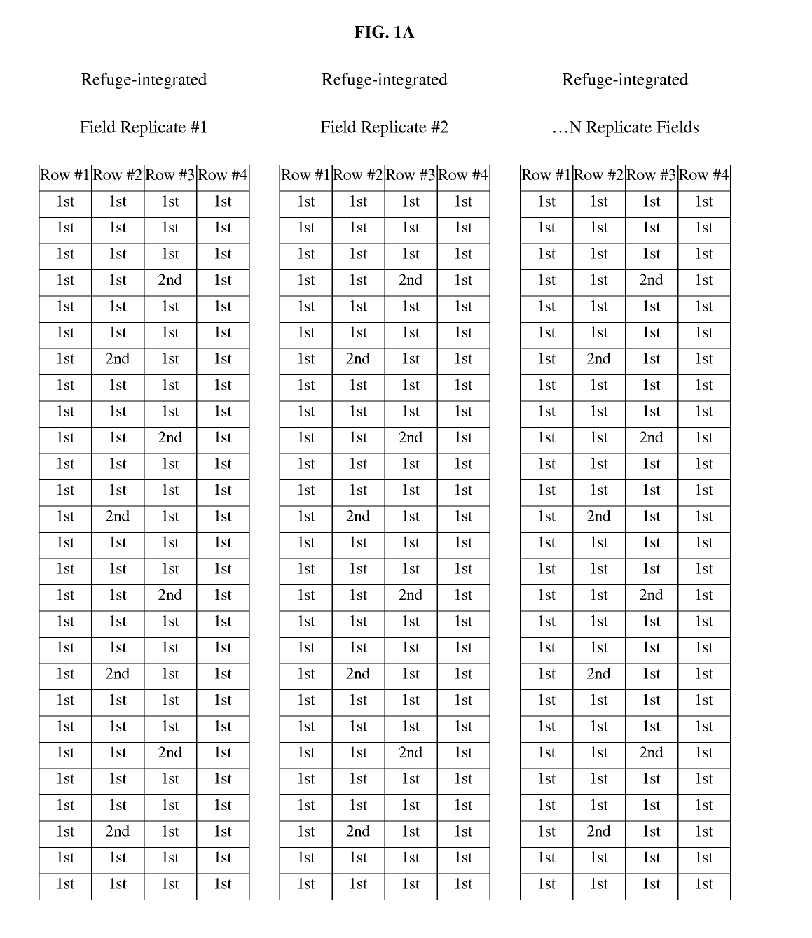

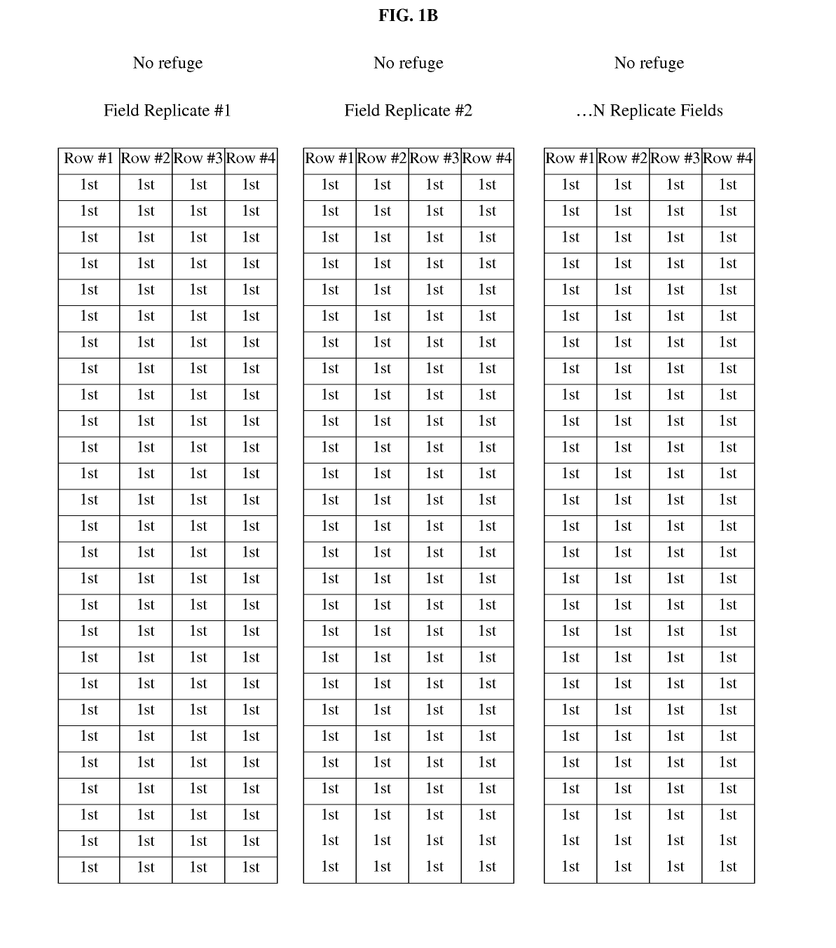

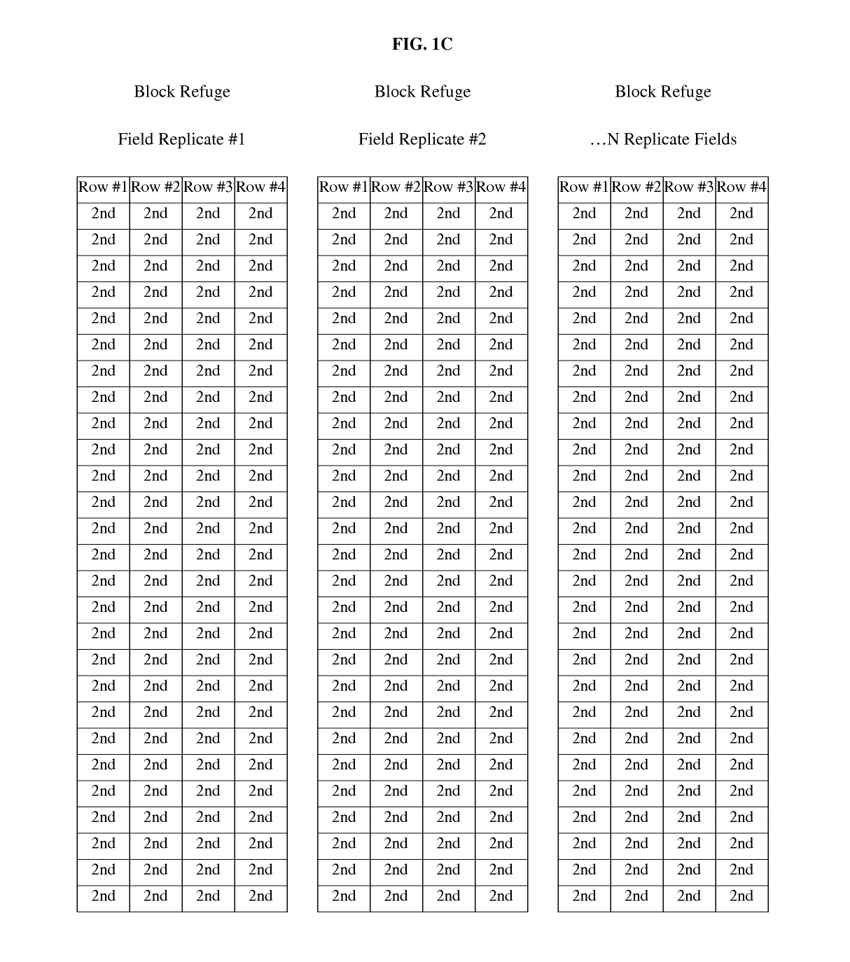

Referring to FIG. 1A, a refuge-integrated field was fabricated having dimensions of 4 rows. Rows were 30 inches apart and seed were planted at a seed drop rate of approximately 34,000 plants per acre, which is equivalent to planting seed approximately every 6 inches within each row. Twenty-five foot (25′) rows were planted which is equivalent to about two hundred first “1^{st}” population maize seed (harboring MON 89034 PIP+PIP#2), and fifteen (15) second “2^{nd}” population maize seed (not harboring the PIPs). The “2^{nd}” population maize seed were systematically planted throughout the field as shown (FIG. 1A). This configuration was replicated four (4) times (approximately 800 plants total).

Referring to FIG. 1B, a PIP-only field was planted having the identical configuration and size as the refuge-integrated field described in this example, with the exception that all seed were first “1^{st}” population maize seed (harboring MON 89034 PIP+PIP#2). This configuration was replicated four (4) times.

Referring to FIG. 1C, a refuge-only field was planted having the identical configuration and size as the refuge-integrated and the PIP-only fields described in this example, with the exception that all seed were second “2^{nd}” population maize seed (not harboring the PIPs). This configuration was replicated four (4) times. These refuge-only fields model a block or structured refuge when accompanying a PIP-only field.

For this example, first “1^{st}” population PIP seed type and second “2^{nd}” population refuge seed type were both rated to (a) reach physiological maturity at about the same time (RMs=110 days), and (b) reach reproductive maturity at about the same time (1312 GDUs to P50). Seed from both “1^{st}” and “2^{nd}” populations were planted at the same time in each field, and fields were comparably managed.

When corn silks began emerging on the primary ear, the ears of about twelve to fourteen plants selected evenly across the replicated fields were infested with lab-reared, MON 89034-susceptible and PIP#2-susceptible Helicoverpa zea (corn earworm, CEW) neonates. For the refuge-only and refuge-integrated fields, only the ears of the plants grown from the second “2^{nd}” population refuge seed type were infested. About one hundred fifty-six (156) plants distributed across the twelve (12) replicated fields were infested in this manner. Ear damage on refuge plants was rated across all replicated fields and larval survival was assessed prior to pupation or prior to freeze, whichever came first.

Of the fifty (50) selected ears infested with CEW neonates in the replicated PIP-only fields, 100% mortality was observed, and no ear damage due to larval feeding was observed (See Table 2 (A)).

Of the fifty (50) selected ears infested with CEW neonates in the replicated refuge-only fields, thirty-seven (37) surviving larvae were observed and ear damage due to larval feeding was observed (See Table 2 (B)). Surviving single CEW larva was observed on each of thirty-seven (37) ears.

Of the fifty-six (56) refuge ears infested with CEW neonates in the replicated refuge-integrated fields, only six (6) surviving corn earworm larvae were observed and ear damage due to larval feeding was negligible (See Table 2 (C)).

The refuge of a refuge-integrated field grown from a standard RIB blend is less effective than the refuge of a refuge-only field.

### Example 2

This example illustrates PIP field trials for evaluating differential maturing refuge, and methods of deploying such refuge.

The refuge-integrated fields of Example 1 (FIG. 1A), illustrated a maize refuge of plants interspersed among a PIP crop population, where the refuge plant population and the PIP plant population reproductively matured contemporaneously as would occur in a field planted from a standard RIB seed blend. The refuge-integrated fields were not as effective at producing susceptible insects when compared to the structured (or “block”) refuge-only fields (Table 2). The refuge-integrated fields and refuge-only fields of Example 1 were used as controls for this example.

Test differential maturing refuge-integrated fields were deployed at the same time and within the same geography as of the fields of Example 1. Four replicates of each test was deployed using the same configuration as that used in Example 1 (FIG. 1A). Each replicate contained approximately two hundred first “1^{st}” population toxin maize seed (harboring MON 89034 PIP+PIP#2) and fourteen (14) second “2^{nd}” population refuge maize seed (not harboring the PIPs). Second “2^{nd}” population refuge maize seed were systematically planted throughout each replicate field as illustrated in FIG. 1A.

Four differential maturing refuge-integrated fields were trialed, each differed in the differential reproductive maturity ratings (ΔRpM) between refuge and PIP seed (Table 1).

TABLE 1
Test seed selected for differential maturing (ΔRpMs)
refuge-integrated fields

| Toxin (PIP) seed | Refuge seed | | | |
|---|---|---|---|---|
| RM₁^{a} | RM₂ᵢ^{a} | | | |
| and | and | ΔRM^{c} | ΔRpM^{d} | |
| RpM₁^{b} | RpM₂ᵢ^{b} | (days) | (GDUs) | Test ID |
| 110 days | 110 and 1312 | 0 | 0 | Control (C) |
| and | 96 and 1267 | −14 | −45 | Test #1 (D) |
| 1312 GDUs | 102 and 1280 | −8 | −32 | Test #2 (E) |
| | 112 and 1335 | +2 | +23 | Test #3 (F) |
| | 117 and 1380 | +7 | +68 | Test #4 (G) |

^{a}RM is the relative maturity rating (days). RM₁ is of 1^{st} population PIP crop. RM₂ᵢ is of 2^{nd} population refuge crop (for each i).
^{b}RpM is the reproductive maturity rating, or “sexual maturity rating”, estimated as the accumulated growing degree units from planting to P50, where P50 means 50% of the population of the maize plants in the field are expected to have started shedding pollen.
^{c}ΔRM = RM₂ᵢ − RM₁.
^{d}ΔRpM = RpM₂ᵢ − RpM₁, or “differential sexual maturity rating” when ΔRpM < > 0.

The silking ear of refuge plants in the refuge-integrated fields were infested with CEW neonates, survival of CEW was assessed, and ear damage was rated in the same manner as described in Example 1 for refuge-integrated fields.

TABLE 2
Results table

| | | Average Ear | Total larval | Fold survival |
|---|---|---|---|---|
| | | damage | survival across | across selected |
| | Description | (cm²)^{a} | selected ears^{a} | ears^{b} |
| Controls | (A) PIP-only | 0 | 0 | NA |
| | (B) Refuge-only | 0.66 | 37 survivors | 6 |
| | (C) Standard refuge-integrated field | 0.05 | 6 survivors | 1 |
| Differential | (D) Test #1, ΔRpM of −45 GDUs | 0.33 | 28 survivors | 5 |
| maturing refuge | (E) Test #2, ΔRpM of −32 GDUs | 0.32 | 26 survivors | 4 |
| | (F) Test #3, ΔRpM of +23 GDUs | 0.57 | 47 survivors | 8 |
| | (G) Test #4, ΔRpM of +68 GDUs | 0.38 | 36 survivors | 6 |

^{a}For tests or controls having refuge plants, approximately 50 to 56 refuge ears were infested and rated. For the PIP-only control, approximately 50 PIP ears were infested.
^{b}For tests or controls having refuge plants, fold survival was compared to the 6 survivors of the standard refuge-integrated field.

The fifty-six (56) refuge ears infested with corn earworm (CEW) neonates in the refuge-integrated fields rated at a ΔRpM of −45 GDUs ((D) Test #1) exhibited about a 5-fold increase in surviving CEW larvae compared to control.

The fifty-six (56) refuge ears infested with CEW neonates in the refuge-integrated fields rated at a ΔRpM of +68 GDUs ((G) Test #4) exhibited about a 6-fold increase in surviving CEW larvae compared to control.

The fifty-six (56) refuge ears infested with CEW neonates in the refuge-integrated fields rated at a ΔRpM of −32 GDUs ((E) Test #2) exhibited about a 4-fold increase in surviving CEW larvae compared to control.

The fifty-six (56) refuge ears infested with CEW neonates in the refuge-integrated fields rated at a ΔRpM of +23 GDUs ((F) Test #3) exhibited about a 8-fold increase in surviving CEW larvae compared to control.

By using ΔRpM to select refuge seed and PIP seed, it was demonstrated that even a ΔRpM of +23 GDUs resulted in an increase in larval survivorship on refuge plant ears compared to no ΔRpM.

A differential maturing refuge is effective when accompanying a PIP crop field when the field comprises plants grown from a blend of PIP seed and refuge seed, where PIP seed and refuge seed are of the same species, where the PIP seed are rated to reproductively mature substantially contemporaneously (RpM₁) in the field, and where the refuge seed are rated to reproductively mature (a) earlier, (b) later, or (c) a combination of earlier and later (RpM₂ᵢ) than the PIP seed as illustrated by the equation:

[(RpM₂₍ᵢ₎−(RpM₁)]|>23 GDUs,

where RpM₁ is the reproductive maturity rating of the PIP seed, where RpM₂₍ᵢ₎ is the reproductive maturity rating for each i population (1, 2 . . . N) of the refuge seed, where for each i, ΔRpM₍ᵢ₎=[(RpM₂₍ᵢ₎−(RpM₁)].

The differential maturing refuge accompanying PIP crop fields exemplified in this example resulted in an increase in surviving insects, thereby decreasing the likelihood for development of pest resistance compared to that of the standard refuge-integrated field. This example illustrated a differential maturing refuge and methods to deploy such.

A refuge crop grown from seed blend containing differential maturing refuge (a) is more effective at delaying evolution of PIP-resistance in kernel-feeding pests when compared to a standard refuge-integrated field where refuge and PIP plants mature contemporaneously, and (b) will be more effective at sustaining PIP product durability.

### Example 3

This example illustrates the potential of a seed treatment for delaying reproductive maturity.

The growth regulator known as s-abscisic acid (s-ABA) can delay seed germination. It is the active ingredient of a product known as BIONIK, offered by Valent BioScience Corporation (VBC). A number of seed from six different corn inbred lines were treated with BIONIK at two concentrations each, “VBS low” and “VBS high”. A number of seed were also left untreated as control. Seed were planted in 4 replicate plots by 5 locations by two different planting dates. Each plot was 2 rows by 20 feet on 30 inch spaced rows. The average of accumulated GDU to P10 were measured for each line. Compared to the untreated control, the average delta GDU for plants grown from VBS low and VBS high treated seed exhibited statistically significant (α=0.050) overall delays to 10% pollen shed of 26 GDUs and 42 GDUs, respectively. This trend held consistent across different locations, across the different inbreds, and across the two different planting dates. Delay to P50 and P90 were also measured and trend was also consistent.

### Example 4

This example illustrates seed treatments for providing differential RpM seed blends, methods of planting such seed blends, and assessing the refuge potential of such seed blends.

About 150,000 toxin hybrid corn seed (harboring MON 89034 PIP+PIP#2) are treated with VBS high. About 12,000 refuge hybrid corn seed are left untreated. Toxin and refuge seed are otherwise rated to mature contemporaneously without treatment of BIONIK. Treated toxin seed and untreated refuge seed are gently blended together to provide improved refuge-in-a-bag (iRIB) seed blends. A “90:10” iRIB seed blend and a “95:5” iRIB seed blend are made. Ratios are toxin:refuge by seed weight. Each bag contains about 80,000 seed.

About 150,000 toxin hybrid corn seed (harboring MON 89034 PIP+PIP#2) and about 12,000 refuge hybrid seed are provided, and serve as untreated controls. These seed come from the same hybrid batch as above and are rated to mature contemporaneously. Untreated toxin and untreated refuge seed are gently blended together to provide RIB seed blends. A “90:10” RIB and a “95:5” RIB are made. Ratios are toxin:refuge by seed weight. Each bag contains about 80,000 seed.

Seed from each bag are planted. Refuge hybrid plants grown from the iRIB seed blends sexually mature earlier than the toxin hybrid plants. Refuge and toxin hybrid plants from the RIB seed blends sexually mature contemporaneously. Insect infestation occurs by hand-infestation or through natural pressure.

Ears on the refuge hybrid plants grown from the iRIB seed blends exhibit fewer toxin seed than ears on the refuge hybrid plants from the RIB seed blends. Ears on the refuge hybrid plants grown from the iRIB seed blend exhibit more insect survivors than ears on the refuge hybrid plants from the RIB seed blends. This example illustrates a differential maturing refuge and methods to deploy such, where refuge crop grown from an iRIB seed blend is (a) more effective at delaying evolution of PIP-resistance in kernel-feeding pests when compared to a refuge-integrated field where refuge and PIP plants mature relatively contemporaneously, and (b) will be more effective at sustaining PIP product durability.

### Example 5

This example illustrates seed treatments for providing differential RpM seed blends, methods of planting such seed blends, and assessing the refuge potential of such seed blends.

About 150,000 toxin hybrid corn seed (harbors MON 89034 PIP+PIP#2) are provided. About 12,000 refuge hybrid corn seed are treated with VBS high using the method of Example 4. Toxin and refuge seed are otherwise rated to mature contemporaneously without treatment of BIONIK. Untreated toxin seed and treated refuge seed are gently blended together to provide improved refuge-in-a-bag (iRIB) seed blends. A “90:10” iRIB seed blend and a “95:5” iRIB seed blend are made. Ratios are toxin:refuge by seed weight. Each bag contains about 80,000 seed.

Bags of “90:10” RIB and a “95:5” RIB are made as in Example 3 as untreated controls, where each bag contains about 80,000 seed.

Seed from each bag are planted. Refuge hybrid plants grown from the iRIB seed blends sexually mature later than the toxin hybrid plants. Refuge and toxin hybrid plants from the RIB seed blends sexually mature contemporaneously.

Ears on the refuge hybrid plants grown from the iRIB seed blends exhibit fewer toxin seed than ears on the refuge hybrid plants from the RIB seed blends.

### Example 6

This example illustrates PIP field trials with contemporaneous maturing refuge and with differential maturing refuge, and methods of deploying differential maturing refuge.

Differential maturing refuge-integrated fields were designed to test varying degrees of differential maturing refuge. ΔRpMs from −89 GDUs to +143 GDUs were tested. Trials were conducted in similar fashion as described in Examples 1 and 2. The toxin seed harbors MON 89034 PIP and PIP#2 (both event PIPs are toxic to CEW).

Seed were selected and planted contemporaneously to create test refuge-integrated and control fields (Table 3), and all fields managed comparably and deployed in similar geography.

TABLE 3
Test seed selected for differential maturing (ΔRpMs)
refuge-integrated fields

| Toxin | | | |
|---|---|---|---|
| (PIP) | Refuge | | |
| Seed | seed | ΔRpM | |
| RpM₁^{a} | RpM₂ᵢ^{a} | seed^{b} | |
| (GDU) | (GDU) | (GDU) | Description |
| NA | 1312 | NA | Refuge-only seed |
| | | | Control |
| 1312 | NA | NA | PIP-only seed |
| | | | Control |
| | 1312 | 0 | Standard refuge-integrated |
| | | | seed control |
| | 1223 | −89 | Differential maturing refuge- |
| | | | integrated seed (Test #1) |
| | 1400 to 1450 | +88 to +138 | Differential maturing refuge- |
| | | | integrated seed (Test #2) |
| | 1420 | +108 | Differential maturing refuge- |
| | | | integrated seed (Test #3) |
| | 1455 | +143 | Differential maturing refuge- |
| | | | integrated seed (Test #4) |

^{a}RpM is the reproductive maturity rating, or “sexual maturity rating”, estimated as the accumulated growing degree units from planting to P50, where 50% of the population of maize plants in the field is expected to have started shedding pollen.
^{b}ΔRpM = RpM₂ᵢ − RpM₁, or “differential sexual maturity rating”, or “differential reproductive maturity rating”.

The ear of refuge plants and ear of PIP plants were infested with CEW neonates when green silks emerged on the ear. The actual differential in sexual maturation between refuge plants and PIP plants in each field was measured. The number of ears exhibiting surviving CEW was counted prior to pupation, and ear damage was measured (Table 5). Ears from plants not infested with CEW were harvested in order to measure cross-pollination of refuge kernels by PIP pollen.

CEW survival is higher and ear damage is higher in fields with differential maturing refuge when compared to the standard refuge-integrated fields. Cross-pollination of refuge kernels by PIP pollen is reduced in fields with differential maturing refuge when compared to the standard refuge-integrated fields.

The differential maturing refuge accompanying PIP crop fields exemplified in this example produced elevated levels of surviving insects compared to a standard refuge-integrated field having no differential maturing refuge. This example illustrated a differential maturing refuge and methods to deploy such.

A refuge crop grown from the seed blend of the present invention (a) is more effective at delaying evolution of PIP-resistance in kernel-feeding pests when compared to a standard refuge-integrated field where refuge and PIP plants mature contemporaneously, and (b) will be more effective at sustaining PIP product durability.

What is claimed is:

1. A corn seed blend comprising:
(a) a first corn seed type comprising a first reproductive maturity rating and a transgene; and
(b) a second corn seed type comprising a second reproductive maturity rating different from the first reproductive maturity rating that lacks said transgene,
wherein the second reproductive maturity rating differs from the first sexual maturity rating by at least 3 days.

2. The corn seed blend of claim 1, wherein plants grown from said second corn seed type are substantially unpollinated by plants grown from said first corn seed type.

3. The corn seed blend of claim 1, wherein plants grown from said first corn seed type are substantially unpollinated by plants grown from said second corn seed type.

4. The corn seed blend of claim 1, wherein a corn ear produced by plants grown from said second corn seed type exhibit a reduced number of seed comprising said transgene when compared to a control corn seed blend comprising seed types rated to reproductively mature contemporaneously.

5. The corn seed blend of claim 1, wherein the ratio of said first corn seed type to said second corn seed type is selected from the group consisting of from about 70:30 to about 99:1, from about 80:20 to about 97:3, and from about 90:10 to about 95:5.

6. The corn seed blend of claim 1, wherein said first corn seed type comprises a transgenic event selected from the group consisting of DP-186165-2, DP-186169-6, DP-187156-3, DP 4114, MIR162, MIR 604, Bt 176, TC1507, DAS-06275-8, DAS-59122-7, Bt11, 5307, MON810, MON89034, MON88017, ZM_S295399, MON87411, MON853, and MON863.

7. The corn seed blend of claim 1, wherein said first corn seed type comprises a transgene encoding for a pesticidal product selected from the group consisting of VIP3A, VIP3Aa, Cry1A.105, Cry2Ab, Cry1F, Cry1A, Cry1Ab, Cry1Ac, Cry34/35, Cry34/35Ab1, Cry34Ab1, Cry35Ab1, Cry3A, mCry3A, eCry3.1Ab, Cry3Bb, Dv49 dsRNA, and Dv_Snf7o.

8. A field of corn plants grown from the corn seed blend of claim 1.

9. A method of reducing the incidence of pollination between plants grown from different seed types in a corn seed blend, the method comprising:
(a) providing a corn seed blend comprising (i) a first corn seed type comprising a first reproductive maturity rating and a transgene and (ii) a second corn seed type comprising a second reproductive maturity rating different from the first reproductive maturity rating that lacks said transgene; and
(b) planting the corn seed blend,
wherein the second reproductive maturity rating differs from the first sexual maturity rating by at least 3 days.

10. The method of claim 9, wherein plants grown from said second corn seed type are substantially unpollinated by plants grown from said first corn seed type.

11. The method of claim 9, wherein plants grown from said first corn seed type are substantially unpollinated by plants grown from said second corn seed type.

12. The method of claim 9, wherein a corn ear produced by plants grown from said second corn seed type exhibit a reduced number of seed comprising said transgene when compared to a control corn seed blend comprising seed types rated to reproductively mature contemporaneously.

13. The method of claim 9, wherein the ratio of said first corn seed type to said second corn seed type in said corn seed blend is selected from the group consisting of from about 70:30 to about 99:1, from about 80:20 to about 97:3, and from about 90:10 to about 95:5.

14. The method of claim 9, wherein said first corn seed type comprises a transgenic event selected from the group consisting of DP-186165-2, DP-186169-6, DP-187156-3, DP 4114, MIR162, MIR 604, Bt 176, TC1507, DAS-06275-8, DAS-59122-7, Bt11, 5307, MON810, MON89034, MON88017, ZM_S295399, MON87411, MON853, and MON863.

15. The method of claim 9, wherein said first corn seed type comprises a transgene encoding for a pesticidal product selected from the group consisting of VIP3A, VIP3Aa, Cry1A.105, Cry2Ab, Cry1F, Cry1A, Cry1Ab, Cry1Ac, Cry34/35, Cry34/35Ab1, Cry34Ab1, Cry35Ab1, Cry3A, mCry3A, eCry3.1Ab, Cry3Bb, Dv49 dsRNA, and Dv_Snf7o.

16. The method of claim 15, wherein said pesticidal agent is active against Helicoverpa zea, and wherein said reduction occurs at the tip portion of the corn ears.

17. A method of reducing the incidence of pollination between plants grown from different seed types in a corn seed blend, the method comprising:
(a) providing a corn seed blend comprising a first corn seed type comprising a transgene and a second corn seed type that lacks said transgene; and
(b) planting the corn seed blend,
wherein said first or said second corn seed type is treated with a formulation to delay or to accelerate the time to reproductive maturation of plants grown from the treated seed.

18. A method of reducing the incidence of pollination between plants grown from different seed types in a corn seed blend comprising applying a treatment to plants grown from said corn seed blend, wherein
(a) plants grown from said first corn seed type comprise a gene or gene product that interacts with said treatment and plants grown from said second corn seed type lack said gene or gene product, and wherein said interaction delays or accelerates time to reproductive maturity of plants; or
(b) plants grown from said second corn seed type comprise a gene or gene product that interacts with said treatment and plants grown from said first corn seed type lack said gene or gene product, and wherein said interaction delays or accelerates time to reproductive maturity of plants.

19. A method for ensuring compliance with government regulations for planting a refuge crop with or alongside of a transgenic crop, said method comprising:
(a) providing a corn seed blend comprising (i) a first corn seed type comprising a first reproductive maturity rating and a transgene and (ii) a second corn seed type comprising a second reproductive maturity rating different from the first reproductive maturity rating that lacks said transgene; and
(b) planting the corn seed blend,
wherein the second reproductive maturity rating differs from the first sexual maturity rating by at least 3 days.

20. The method of claim 19, wherein a corn ear produced by plants grown from said second corn seed type exhibit a reduced number of seed comprising said transgene when compared to a control corn seed blend comprising seed types rated to reproductively mature contemporaneously.
